SEQUENCE LISTING (11 sequences)

SEQ ID NO: 1 - protein, 377 aa, Synthetic construct
  REGION 1..377: note = reference with X
  VARIANT 23: note = X means R or V or A or L or K or Q or T or G
  VARIANT 262: note = X means R or V or A or L or K or Q or T or G
  VARIANT 264: note = X means R or V or A or L or K or Q or T or G
  VARIANT 298: note = X means R or V or A or L or K or Q or T or G
MASSHHHHHHSSGSEKKISEYAXQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQXDXLDHFQKCFLILKLHHQRVDSSKSNQQEGKTWKAXRVDLVMCPYENRAFALLGWTGSPQFNRDLRRYATHERKMMLDNHALYDKTKRVFLEAESEEEIFAHLGLDYIEPWERNA

SEQ ID NO: 2 - protein, 377 aa, Synthetic construct
  REGION 1..377: note = reference
MASSHHHHHHSSGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 3 - protein, 377 aa, Synthetic construct
  REGION 1..377: note = T264R + I298V
MASSHHHHHHSSGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDRLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

SEQ ID NO: 4 - protein, 377 aa, Synthetic construct
  REGION 1..377: note = C23A + T264R + I298V
MASSHHHHHHSSGSEKKISEYAAQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDRLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

SEQ ID NO: 5 - protein, 377 aa, Synthetic construct
  REGION 1..377: note = V262A + I298V
MASSHHHHHHSSGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQADTLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

SEQ ID NO: 6 - protein, 19 aa, Synthetic construct
  REGION 1..19: note = Histidine tag
MASSHHHHHHSSGSEKKIS

SEQ ID NO: 7 - protein, 358 aa, Synthetic construct
  REGION 1..358: note = reference with X
  VARIANT 4: note = X means R or V or A or L or K or Q or T or G
  VARIANT 243: note = X means R or V or A or L or K or Q or T or G
  VARIANT 245: note = X means R or V or A or L or K or Q or T or G
  VARIANT 279: note = X means R or V or A or L or K or Q or T or G
EYAXQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQXDXLDHFQKCFLILKLHHQRVDSSKSNQQEGKTWKAXRVDLVMCPYENRAFALLGWTGSPQFNRDLRRYATHERKMMLDNHALYDKTKRVFLEAESEEEIFAHLGLDYIEPWERNA

SEQ ID NO: 8 - protein, 358 aa, Synthetic construct
  REGION 1..358: note = reference
EYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 9 - protein, 358 aa, Synthetic construct
  REGION 1..358: note = T245R + I279V
EYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDRLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

SEQ ID NO: 10 - protein, 358 aa, Synthetic construct
  REGION 1..358: note = C4A + T245R + I279V
EYAAQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQVDRLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

SEQ ID NO: 11 - protein, 358 aa, Synthetic construct
  REGION 1..358: note = V243A + I279V
EYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYNLVESTFEKFKLPSRQADTLDHFQKCFLILKLHHQRVDSSKSNQQEGKTWKAVRVDLVMCPYENRAFALLGWTGSPQFNRDLRRYATHERKMMLDNHALYDKTKRVFLEAESEEEIFAHLGLDYIEPWERNA

(19) United States
(12) Patent Application Publication
(10) Pub. No.: US 2025/0034532 A1
(43) Pub. Date: Jan. 30, 2025

(54) NOVEL TERMINAL DEOXYNUCLEOTIDYL TRANSFERASE (TdT) VARIANT AND USES THEREOF

(71) Applicant: DNA Script, Le Kremlin-Bicêtre (FR)

(72) Inventors: Daniel MCHALE, Le Kremlin-Bicêtre (FR); Florent DEGROTTE, Le Kremlin-Bicêtre (FR); Faten JAZIRI, Le Kremlin-Bicêtre (FR); Mikhael SOSKINE, Le Kremlin-Bicêtre (FR); Elodie SUNE, Le Kremlin-Bicêtre (FR); Elise CHAMPION, Le Kremlin-Bicêtre (FR)

(21) Appl. No.: 18/709,292

(22) PCT Filed: Nov. 10, 2022
(86) PCT No.: PCT/EP2022/081547

§ 371 Date: May 10, 2024

(30) Foreign Application Priority Data
Nov. 10, 2021 (FR) FR2111980

(51) Int. Cl. C12N 9/12; C12P 19/34

(52) U.S. Cl. CPC C12N 9/1264; C12P 19/34

(57) ABSTRACT

The invention states to a novel DNA polymerase of the polX family, in particular a Terminal deoxynucleotidyl Transferase (TdT) variants comprising specific mutations or substitutions and their uses.

### TECHNICAL FIELD

The invention relates to a novel DNA polymerase of the polX family, a Terminal deoxynucleotidyl Transferase (TdT) variant comprising at least one specific mutation or substitution and its use.

### BACKGROUND

The current synthetic biology has been a rapidly growing field of research in recent years. Also, the synthetic biology offers the possibility to synthesize biological material. Therefore, biological material that do not exist in nature can be obtained, which provide therapeutics or diagnostics solutions, for example genomic and diagnostic sequencing, multiplex nucleic acid amplification, therapeutic antibody development, synthetic biology, nucleic acid-based therapeutics, DNA origami, DNA-based data storage, and the like.

Gene synthesis is usually done by chemically based synthesis methods. But those methods present several problems. As an example, for each added nucleotide, the probability of genetic error is about 0.5% and the longer the sequence, the higher the probability of containing errors. Recently, interest has arisen in supplementing or replacing chemically-based synthesis methods by enzymatically-based methods using template-free polymerases, such as, terminal deoxynucleotidyl transferase (TdT), because of the proven efficiency of such enzymes, high synthesis rate, limited risk of error and the benefit of mild non-toxic reaction conditions, e.g. Ybert et al, International patent publication WO2015/159023; Hiatt et al, U.S. Pat. No. 5,763,594; Jensen et al, Biochemistry, 57:1821-1832 (2018); and the like.

Most methods using enzyme-based synthesis require the use of reversibly blocked nucleoside triphosphates in order to obtain a desired sequence in the polynucleotide product. Unfortunately, template-free polymerases incorporate such modified nucleoside triphosphates with reduced efficiency as compared to unmodified nucleoside triphosphates. Thus, new template-free polymerases variants with better incorporation efficiencies for modified nucleoside triphosphates has been developed, e.g. Champion et al, U.S. patent publication US2019/0211315 and then International patent publication WO2021116270; Ybert et al, International patent publication WO2017/216472, and the like.

Despite the development of new template-free polymerases variants, the DNA quality during enzymatic DNA synthesis could be improved, for example, by reducing the risk of lack of incorporation of the desired nucleotide such as substitutions, deletions, or insertions during the DNA synthesis. At least in part, these deletions are caused by the inability of template-free polymerases to incorporate the desired nucleotide, in particular when the primer has a hairpin structure.

Thus, there is a need for new template-free polymerases variants to limit or prevent the number of substitutions or deletions during the DNA synthesis and thus achieve a satisfying DNA quality synthesis.

To address this need, the inventors has developed new TdT variants and surprisingly discovered that more active TdT variants produce DNA strand or RNA strand with less misincorporations such as deletions, insertions or substitutions, particularly during DNA synthesis.

### SUMMARY OF THE INVENTION

Thus, the invention relates to a novel terminal deoxynucleotidyl transferase (TdT) variant with or without a linker moiety which may have a sequence set forth in SEQ ID NO:6 as an example.

The novel terminal deoxynucleotidyl transferase (TdT) variant comprises an amino acid sequence at least 70% identical to SEQ ID NO: 2, preferably at least 75%, 80%, 85%, 90%, 95%, 96%, 97%, 98%, 99% identical to SEQ ID No. 2 and optionally less than 100% identical to SEQ ID No. 2, wherein said amino acid sequence comprises at least one amino acid substitution with a substitute amino acid at position selected from a first group consisting of positions 23, 262, 264 and 298, or at functionally equivalent position of each position of said first group, wherein the positions are numbered by reference to the amino acid sequence set forth in SEQ ID NO: 2 or an amino acid sequence at least 70% identical to SEQ ID NO: 8, preferably at least 75%, 80%, 85%, 90%, 95%, 96%, 97%, 98%, 99% identical to SEQ ID No. 8 and optionally less than 100% identical to SEQ ID No. 8, wherein said amino acid sequence comprises at least one amino acid replacement with a replacing amino acid at position selected from a second group consisting of positions 4, 243, 245 and 279, or at functionally equivalent position of each position of said second group, wherein the positions are numbered by reference to the amino acid sequence set forth in SEQ ID NO: 8.

In some embodiments, the novel TdT variant comprises the amino acid sequence as set forth in SEQ ID NO: 2, wherein said amino acid sequence comprises at least one amino acid substitution with an amino acid at position selected from the group consisting of positions 23, 262, 264 and 298, wherein the positions are numbered by reference to the amino acid sequence set forth in SEQ ID NO: 2.

In some embodiments, the novel TdT variant comprises the amino acid sequence as set forth in SEQ ID NO: 8, wherein said amino acid sequence comprises at least one amino acid substitution with an amino acid at position selected from the group consisting of positions 4, 243, 245 and 279, wherein the positions are numbered by reference to the amino acid sequence set forth in SEQ ID NO: 8.

In another embodiment, the TdT variant comprises the amino acid sequence as set forth in SEQ ID NO: 1, wherein said amino acid sequence comprises at least one amino acid substitution with another amino acid at position selected from the group consisting of positions 23, 262, 264 and 298, or at functionally equivalent position of each position of said group, wherein the positions are numbered by reference to the amino acid sequence set forth in SEQ ID NO: 1. In particular, said substituted amino acid is indicated by “X” in SEQ ID NO: 1.

In another embodiment, the TdT variant comprises the amino acid sequence as set forth in SEQ ID NO: 7, wherein said amino acid sequence comprises at least one amino acid replacement with a replacing amino acid at position selected from the group consisting of positions 4, 243, 245 and 279, or at functionally equivalent position of each position, wherein the positions are numbered by reference to the amino acid sequence set forth in SEQ ID NO: 7. In particular, said replaced amino acid is indicated by “X” in SEQ ID NO: 7.

In a particular embodiment the terminal deoxynucleotidyl transferase (TdT) variant comprises more than one amino acid substitution in the amino acid sequence as set forth in SEQ ID NO: 1 or SEQ ID NO: 2, preferably at least two amino acid substitutions, more preferably at least three amino acid substitutions.

In a particular embodiment the terminal deoxynucleotidyl transferase (TdT) variant comprises more than one amino acid replacement in the amino acid sequence as set forth in SEQ ID NO: 7 or SEQ ID NO: 8, preferably at least two amino acid replacements, more preferably at least three amino acid replacements.

In some embodiments, the amino acid substituted in the amino acid sequence as set forth in SEQ ID NO: 1 or SEQ ID NO: 2 is selected from the group consisting of Arginine (R), Valine (V), Alanine (A), Leucine (L), Lysine (K), Glutamine (Q), Threonine (T) and Glycine (G).

In some embodiments, the amino acid replaced in the amino acid sequence as set forth in SEQ ID NO: 7 or SEQ ID NO: 8 is selected from the group consisting of Arginine (R), Valine (V), Alanine (A), Leucine (L), Lysine (K), Glutamine (Q), Threonine (T) and Glycine (G).

In a particular embodiment, the TdT variant comprises the amino acid sequence selected from SEQ ID NO: 3, SEQ ID NO: 4 and SEQ ID NO: 5.

In a particular embodiment, the TdT variant comprises the amino acid sequence selected from SEQ ID NO: 9, SEQ ID NO: 10 and SEQ ID NO: 11.

The invention also relates to kits for performing template-free polynucleotide elongations of any predetermine sequence, wherein the kits include at least one TdT variant of the invention, at least one 3′-O-modified nucleoside triphosphates, and optionally at least one initiator. Such kit may further comprise a deoxyribonucleoside triphosphates (dNTPs) for A, C, G and T for DNA elongation, or ribonucleoside triphosphates (rNTPs) for rA, rC, rG and U for RNA elongation.

Thus, the kit comprises at least one TdT variant according to the invention. In this context, one understands that the kit can comprise one TdT variant or many TdT variants, for example two different TdT variants.

In a particular embodiment, the kit comprises more than one TdT variant, in particular the kit comprises two TdT variants according to the invention, more preferably the kit comprises three TdT variants according to the invention.

In some embodiments, the kit comprises at least one TdT variant comprises the amino acid sequence selected from SEQ ID NO: 3, SEQ ID NO: 4 and SEQ ID NO: 5. In a particular embodiment the kit comprises two TdT variants, the first TdT variant comprising the amino acid sequence selected from SEQ ID NO: 3, SEQ ID NO: 4 and SEQ ID NO: 5 and the second TdT variant comprising the amino acid sequence selected from SEQ ID NO: 3, SEQ ID NO: 4 and SEQ ID NO: 5, said second TdT variant being different from the first TdT variant. In another particular embodiment, the kit comprises three TdT variants according to the invention, more preferably the kit comprises a TdT variant comprising the amino acid sequence as set forth in SEQ ID NO: 3, a TdT variant comprising the amino acid sequence as set forth in SEQ ID NO: 4 and a TdT variant having the amino acid sequence as set forth in SEQ ID NO: 5.

In some embodiments, the kit comprises at least one TdT variant comprising an amino acid sequence at least 70% identical to an amino acid sequence selected from SEQ ID NO: 3, SEQ ID NO: 4 and SEQ ID NO: 5. In a particular embodiment the kit comprises two TdT variants, the first TdT variant comprising an amino acid sequence at least 70% identical to an amino acid sequence elected from SEQ ID NO: 3, SEQ ID NO: 4 and SEQ ID NO: 5 and the second TdT variant comprising an amino acid sequence at least 70% identical to an amino acid sequence selected from SEQ ID NO: 3, SEQ ID NO: 4 and SEQ ID NO: 5, said second TdT variant being different from the first TdT variant.

In some embodiments, the kit comprises at least one TdT variant comprising an amino acid sequence at least 70% identical to an amino acid sequence selected from SEQ ID NO: 9, SEQ ID NO: 10 and SEQ ID NO: 11. In a particular embodiment the kit comprises two TdT variants, the first TdT variant comprising an amino acid sequence at least 70% identical to an amino acid sequence elected from SEQ ID NO: 9, SEQ ID NO: 10 and SEQ ID NO: 11 and the second TdT variant comprising an amino acid sequence at least 70% identical to an amino acid sequence selected from SEQ ID NO: 9, SEQ ID NO: 10 and SEQ ID NO: 11, said second TdT variant being different from the first TdT variant.

In some embodiments, the first TdT variant an amino acid sequence at least 70% identical to SEQ ID NO: 3 and the second variant comprises an amino acid sequence at least 70% identical to SEQ ID NO: 5.

In some embodiments, the first TdT variant an amino acid sequence at least 70% identical to SEQ ID NO: 9 and the second variant comprises an amino acid sequence at least 70% identical to SEQ ID NO: 11

Advantageously, the TdT variant according to the invention overcomes the technical problems of prior art TdT variants and prevent deletions, insertions and lowers incorporation of other undesired triphosphates, for example chemically damaged dNTPs, during the enzymatic synthesis of nucleic acids.

The present invention also relates to a method of synthesizing a polynucleotide, the method comprising the steps of:
a. providing at least one initiator having a 3′-terminal nucleotide which has a free 3′-hydroxyl,
b. contacting under elongation conditions said at least one initiator having a free 3′-O-hydroxyl with a 3′-O-blocked nucleoside triphosphate and a TdT variant according to the invention, so that said at least one initiator is elongated by incorporation of a 3′-O-blocked nucleoside triphosphate to form a 3′-O-blocked elongated fragment, and
c. deblocking the elongated fragment to form an elongated fragment having free 3′-hydroxyl, and
d. repeating steps b. and c. by contacting under elongation conditions the elongated fragment obtained in step c., until the polynucleotide is formed.

In further embodiments, the invention includes at least one nucleic acid molecule encoding a TdT variant described above. In other words, the invention can include more than one nucleic acids, each said nucleic acid molecule encoding a protein sequence corresponding to a TdT variant that has been described.

The invention also includes at least one expression vector comprising such nucleic acid molecule, and at least one host cell comprising the aforementioned nucleic acid molecule or the aforementioned expression vector. In still further embodiments, the invention includes method for producing at least one TdT variant of the invention, wherein a host cell is cultivated under culture conditions allowing the expression of the nucleic acid encoding said TdT variant, and wherein the TdT variant is optionally retrieved.

### BRIEF DESCRIPTION OF THE DRAWINGS

FIG. 1 illustrates diagrammatically the steps of a method of template-free enzymatic nucleic acid synthesis using TdT variants of the invention.

FIG. 2 represents Hairpin (top) and Duplex (bottom) iDNA applied to measure activity of TdT.

FIG. 3 represents the activity and synthesis performance of TdT variant having SEQ ID NO:3, or SEQ ID NO: 4 or SEQ ID NO:5, respectively variant having mutation T264R+1298V, or C23A+T264R+1298V, or V262A+1298V. In panel A: Enzymatic rates for +A reaction in TAGCT+A duplex test. In panel B: Enzymatic rates for +G reaction with CAGCAAGGCT+G hairpin.

FIG. 4 represents the average of deletion, insertion, substitution added during the synthesis of 24 sequences by the TdT variant having SEQ ID NO:3, or SEQ ID NO:4 or SEQ ID NO:5, respectively variant having mutation T264R+1298V, or C23A+T264R+1298V, or V262A+1298V, compared to a synthesis performed by the TdT variant of SEQ ID NO:2 (denominated reference).

### DETAILED DESCRIPTION OF THE INVENTION

### Definition

The amino acids are represented in this description by a one-letter or three-letter code according to the following nomenclature: A: Ala (alanine); R: Arg (arginine); N: Asn (asparagine); D: Asp (aspartic acid); C: Cys (cysteine); Q: Gln (glutamine); E: Glu (glutamic acid); G: Gly (glycine); H: His (histidine); I: Ile (isoleucine); L: Leu (leucine); K: Lys (lysine); M: Met (methionine); F: Phe (phenylalanine); P: Pro (proline); S: Ser (serine); T: Thr (threonine); W: Trp (tryptophan); Y: Tyr (tyrosine); V: Val (valine) and also X (undetermined amino acid).

“Terminal deoxynucleotidyl transferase (TdT) variant” in the context of the invention, means a group of TdT mutants that shares a set of mutations or alterations. An alteration or mutation can be a substitution, an insertion and/or a deletion in one or more positions and allowing to preserve a DNA polymerase activity. For example, one or two, or three mutations located at the same amino acid residue position, for example Threonine has been substituted by Lysine at position 28. In particular, TdTs having the amino acid sequence set forth in SEQ ID NO: 3, SEQ ID NO: 4 and SEQ ID NO: 5 are mutants and said TdTs share a set of mutations and constitute a variant.

TdT variant according to the invention is truncated TdT variant. The TdT variant can be obtained by various techniques well known in the art. In particular, examples of techniques for modifying the DNA sequence encoding wild-type proteins include, without being limited thereto, directed mutagenesis, random mutagenesis, and the construction of synthetic polynucleotides.

“Truncated TdT variant”, in the context of the invention, means a TdT which does not comprise the N-terminal part of the corresponding wild-type TdT. TdT variants according to the invention are N-terminally truncated TdTs lacking amino acids residues 1 to 132 of the corresponding wild-type parent NP_001036693.1 [Mus musculus]) TdT sequence.

“Undetermined amino acid” or “unknown amino acid” or “X”, in the context of the invention, mean an amino acid which can be one of the 20 amino acids selected from the group consisting of Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine and Valine.

“Substituted amino acid” or “replaced amino acid” in the context of the invention, means a substitution of an original amino acid (before substitution or mutation) with another amino acid at a specific position, in particular position selected from a first group consisting of positions 23, 262, 264 and 298, which positions are numbered by reference to the amino acid sequence set forth in SEQ ID NO: 2 or a second group consisting of positions 4, 243, 245 and 279, which positions are numbered by reference to the amino acid sequence set forth in SEQ ID NO: 8. For example at position 23 in SEQ ID NO: 2 or 4 in SEQ ID NO: 8, the original amino acid is C and the substituted amino acid is A.

“% of identity” or “percentage of identity” or “at least % identical to” between two nucleic acid or amino acid sequences in the sense of the present invention is understood to designate a percentage of nucleotides or of amino acid residues which are identical between the two sequences to be compared, which is obtained after the best alignment, this percentage being purely statistical and the differences between the two sequences being distributed randomly and over their entire length. The best alignment of the sequences for the comparison can be carried out, besides manually, by means of the local homology algorithm of Smith and Waterman (1981) (Ad. App. Math. 2:482), by means of the local homology algorithm of Neddleman and Wunsch (1970) (J. Mol. Biol. 48:443), by means of the similarity search method of Pearson and Lipman (1988) (Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, Wis.), by means of the online alignment software Mutalin (http://multalin.toulouse.inra.fr/multalin/multalin.html; 1988, Nucl. Acids Res., 16 (22), 10881-10890).

“Functionally equivalent sequence” in the sense of the present invention is understood to mean a sequence of a DNA polymerase of the polX family, in particular TdT having a sequence, i. e. amino acid sequence, of at least 70%, 75%, 80%, 85%, 90%, preferably at least 95%, 97%, 99% of identity to SEQ ID NO: 1 or SEQ ID NO: 2 or SEQ ID NO: 7 or SEQ ID NO: 8 and having an identical functional role.

“Functionally equivalent residue” is understood to mean a residue in a sequence of a DNA polymerase of the polX family having a sequence homologous to SEQ ID NO: 2 or SEQ ID NO: 8 and having an identical functional role. The “functionally equivalent position” is thus the position of the functionally equivalent residue in the homologous sequence.

The functionally equivalent residues are identified using sequence alignments which are carried out, for example, by means of the online alignment software Mutalin (http://multalin.toulouse.inra.fr/multalin/multalin.html; 1988, Nucl. Acids Res., 16 (22), 10881-10890). After alignment, the functionally equivalent residues are in homologous positions on the different sequences considered. The alignments of sequences and the identification of functionally equivalent residues can occur between any DNA polymerases of the polX family and their natural variants, including interspecies variants.

“Comprise at least one amino acid substitution” or “comprising at least one amino acid mutation” in the sense of the present invention is understood to mean that the variant has one or more substitutions or mutations as indicated with respect to the sequence SEQ ID NO: 1 or SEQ ID NO: 2, but it can have other modifications, in particular substitutions, deletions or additions.

“Comprise at least one amino acid replacement” in the sense of the present invention is understood to mean that the variant has one or more substitutions or mutations as indicated with respect to the sequence SEQ ID NO: 7 or SEQ ID NO: 8, but it can have other modifications, in particular substitutions, deletions or additions.

The invention relates to variant of DNA polymerases of the polX family, in particular TdT variants which are stabilized variants of the TdT polymerase that can be used for synthesizing polynucleotides, such as DNA or RNA, without a template strand. The TdT variants of the invention allow modified nucleotides, and more particularly 3′O-reversibly modified nucleoside triphosphates, to be used in an enzyme-based method of polynucleotide synthesis. In particular, the TdT variant is a truncated TdT variant, more particularly a N-terminal truncated TdT variant.

### Template-Free Enzymatic Synthesis

Template-free enzymatic synthesis of polynucleotides may be carried out by a variety of known protocols using template-free polymerases, such as terminal deoxynucleotidyl transferase (TdT), including variants thereof engineered to have improved characteristics, such as greater temperature stability or greater efficiency in the incorporation of 3′-O-blocked deoxynucleoside triphosphates (3′-O-blocked dNTPs), e.g. Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57:1821-1832 (2018); Mathews et al, Organic & Biomolecular Chemistry, DOI: 0.1039/c6ob01371f (2016); Schmitz et al, Organic Lett., 1 (11): 1729-1731 (1999).

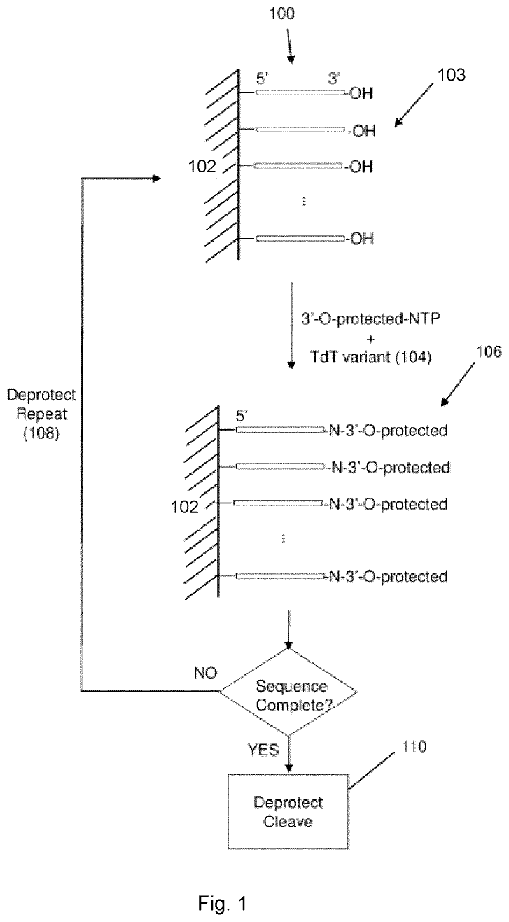

In some embodiments, the method of enzymatic DNA synthesis comprises repeated cycles of steps, such as are illustrated in FIG. 1, in which a nucleotide is added in each cycle. Initiator polynucleotides (100) are provided, for example, attached to solid support (102), which have free 3′-hydroxyl groups (103). To the initiator polynucleotides (100) (or elongated initiator polynucleotides in subsequent cycles) are added a 3′-O-protected-dNTP and a TdT variant under conditions (104) effective for the enzymatic incorporation of the 3′-O-protected-dNTP onto the 3′ end of the initiator polynucleotides (100) (or elongated initiator polynucleotides). This reaction produces elongated initiator polynucleotides whose 3′-hydroxyls are protected (106).

In other words, the method of synthesizing a polynucleotide, comprises the steps of:
a. providing at least one initiator having a 3′-terminal nucleotide having a free 3′-hydroxyl,
b. contacting under elongation conditions said at least one initiator having free 3′-O-hydroxyls with a 3′-O-blocked nucleoside triphosphate and a TdT variant according to the invention, so that the initiator is elongated by incorporation of a 3′-O-blocked nucleoside triphosphate to form a 3′-O-blocked elongated fragment,
c. deblocking the elongated fragment to form elongated fragment having free 3′-hydroxyls, and
d. repeating steps b. and c. by contacting under elongation conditions the elongated fragment obtained in step c., until the polynucleotide is formed.

If the elongated initiator polynucleotide contains a completed sequence, then the 3′-O-protection group is removed, or deprotected, and the desired sequence is cleaved from the original initiator polynucleotide. Such cleavage may be carried out using any of a variety of single strand cleavage techniques, for example, by inserting a cleavable nucleotide or cleavable linker at a predetermined location within the original initiator polynucleotide. Exemplary cleavable nucleotides or linkers include, but are not limited to, (i) a uracil nucleotide which is cleaved by uracil DNA glycosylase; (ii) a photocleavable group, such as a nitrobenzyl linker, as described in U.S. Pat. No. 5,739,386; or an inosine which is cleaved by endonuclease V.

In some embodiments, a cleaved polynucleotide may have a free 5′-hydroxyl; in other embodiments, a cleaved polynucleotide may have a 5′-phosphorylated end. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

In some embodiments, an ordered sequence of nucleotides are coupled to an initiator nucleic acid using a TdT in the presence of 3′-O-reversibly blocked dNTPs in each synthesis step. In some embodiments, the method of synthesizing a polynucleotide comprises the steps of (a) providing an initiator having a free 3′-hydroxyl; (b) reacting under extension conditions the initiator or an extension intermediate having a free 3′-hydroxyl with a TdT in the presence of a 3′-O-blocked nucleoside triphosphate to produce a 3′-O-blocked extension intermediate; (c) deblocking the extension intermediate to produce an extension intermediate with a free 3′-hydroxyl; and (d) repeating steps (b) and (c) until the polynucleotide is synthesized. Sometime “an extension intermediate” is also referred to as an “elongation fragment.”

In some embodiments, an initiator is provided as an oligonucleotide attached to a solid support, e.g. by its 5′ end. The above method may also include washing steps after the reaction, or extension, step, as well as after the de-blocking step. For example, the step of reacting may include a sub-step of removing unincorporated nucleoside triphosphates, e.g. by washing, after a predetermined incubation period, or reaction time. Such predetermined incubation periods or reaction times may be a few seconds, e.g. 30 sec, to several minutes, e.g. 30 min.

The above method may also include capping step(s) as well as washing steps after the reacting, or extending, step, as well as after the deblocking step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions.

In some embodiments that comprise serial synthesis of polynucleotides, capping steps may be undesirable as capping may prevent the production of equal molar amounts of a plurality of polynucleotides. Without capping, sequences will have a uniform distribution of deletion errors, but each of a plurality of polynucleotides will be present in equal molar amounts. This would not be the case where non-extended fragments are capped.

In some embodiments, reaction conditions for an extension or elongation step may comprise the following: 2.0 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes. In embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out in a 50 μL volume, at a temperature within the range of RT to 45° C. for 30 seconds to several minutes.

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: U.S. Pat. Nos. 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl) phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal deblocking conditions. The following exemplary pairs of blocking groups may be used in parallel synthesis embodiments, such as those described above. It is understood that other blocking group pairs, or groups containing more than two, may be available for use in these embodiments of the invention.

| | |
|---|---|
| 3′-O-NH2 | 3′-O-azidomethyl |
| 3′-O-NH2 | 3′-O-allyl |
| 3′-O-NH2 | 3′-O-phosphate |
| 3′-O-azidomethyl | 3′-O-allyl |
| 3′-O-azidomethyl | 3′-O-phosphate |
| 3′-O-allyl | 3′-O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mer-captoethanol, and one Unit T4 polynucleotide kinase. The reaction proceeds for one minute at a temperature of 37° C.

A “3′-phosphate-blocked” or “3′-phosphate-protected” nucleotide refers to nucleotides in which the hydroxyl group at the 3′-position is blocked by the presence of a phosphate containing moiety. Examples of 3′-phosphate-blocked nucleotides in accordance with the invention arc nucleotidyl-3′-phosphate monoester/nucleotidyl-21,3′-cyclic phosphate, nucleotidyl-2′-phosphate monoester and nucleotidyl-2′ or 3′-alkylphosphate diester, and nucleotidyl-2′ or 3′-pyrophosphate. Thiophosphate or other analogs of such compounds can also be used, provided that the substitution does not prevent dephosphorylation resulting in a free 3′-OH by a phosphatase.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148:1-6 (1994); Cameron et al, Biochemistry, 16 (23): 5120-5126 (1977); Rasolonjatovo et al, Nucleosides & Nucleotides, 18 (4&5): 1021-1022 (1999); Ferrero et al, Monatshefte fur Chemie, 131:585-616 (2000); Taunton-Rigby et al, J. Org. Chem., 38 (5): 977-985 (1973); Uemura et al, Tetrahedron Lett., 30 (29): 3819-3820 (1989); Becker et al, J. Biol. Chem., 242 (5): 936-950 (1967); Tsien, International patent publication WO1991/006678.

As used herein, an “initiator” (or equivalent terms, such as, “initiating fragment,” “initiator nucleic acid,” “initiator oligonucleotide,” or the like) refers to a short oligonucleotide sequence with a free 3′-end, which can be further elongated by a template-free polymerase, such as TdT. In one embodiment, the initiating fragment is a DNA initiating fragment. In an alternative embodiment, the initiating fragment is an RNA initiating fragment. In some embodiments, the initiating fragment possesses between 3 and 100 nucleotides, in particular between 3 and 20 nucleotides. In some embodiments, the initiating fragment is single-stranded. In an alternative embodiment, the initiating fragment is double-stranded. In a particular embodiment, an initiator oligonucleotide synthesized with a 5′-primary amine may be covalently linked to magnetic beads using the manufacturer's protocol. Likewise, an initiator oligonucleotide synthesized with a 3′-primary amine may be covalently linked to magnetic beads using the manufacturer's protocol. A variety of other attachment chemistries amenable for use with embodiments of the invention are well-known in the art, e.g. Integrated DNA Technologies brochure, “Strategies for Attaching Oligonucleotides to Solid Supports,” v.6 (2014); Hermanson, Bioconjugate Techniques, Second Edition (Academic Press, 2008); and like references.

Many of the 3′-O-blocked dNTPs employed in the invention may be purchased from commercial vendors or synthesized using published techniques, e.g. U.S. Pat. No. 7,057,026; International patent publications WO2004/005667, WO91/06678; Canard et al, Gene (cited above); Metzker et al, Nucleic Acids Research, 22:4259-4267 (1994); Meng et al, J. Org. Chem., 14:3248-3252 (3006); U.S. patent publication 2005/037991. In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

—O—Z

wherein —Z is any of —C(R′)₂—O—R″, —C(R′)₂—N(R″)₂, —C(R′)₂—N(H) R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′) 2 represents a group of formula ═C (R′″)₂ wherein each R′″ may be the same or different and is selected from the group comprising hydrogen and halogen atoms and alkyl groups, with the proviso that in some embodiments the alkyl of each R′″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In certain embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, —Z of the modified nucleotide or nucleoside is of formula-C(R′)₂—N3. In certain embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, MA)

In a further particular embodiments, the 3′-O-blocked nucleotide triphosphate is blocked by either a 3′-O-azidomethyl, 3′-O—NH₂ or 3′-O-allyl group. In other embodiments, 3′-blocked nucleotide triphosphate is blocked by either a 3′-O-azidomethyl, 3′-O—NH₂.

In some embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

### TdT Variant

According to a first aspect of the invention, the TdT variant according to the invention comprises the amino acid sequence as set forth in SEQ ID NO: 2, wherein said amino acid sequence comprises at least one amino acid substitution with an amino acid at position selected from the group consisting of positions 23, 262, 264 and 298, wherein the positions are numbered by reference to the amino acid sequence set forth in SEQ ID NO: 2. In particular, the TdT variant according to the invention (i) is capable of synthesizing a nucleic acid fragment without a template and (ii) is capable of incorporating a 3′-O-modified nucleotide onto a nucleic acid fragment.

In a particular embodiment, said amino acid sequence can comprise more than one amino acid substitution, preferably at least two, or three selected from the group consisting of positions 23, 262, 264 and 298. Said TdT variants are capable of synthesizing a DNA strand and/or an RNA strand.

In another embodiment, the TdT variant according to the invention has the amino acid sequence as set forth in SEQ ID NO: 1, wherein said amino acid sequence comprises at least one amino acid substitution with an amino acid at position selected from the group consisting of positions 23, 262, 264 and 298, wherein the positions are numbered by reference to the amino acid sequence set forth in SEQ ID NO: 1. Said TdT variant having the amino acid sequence set forth in SEQ ID NO: 1 comprises at least one undetermined amino acid at position selected from the group consisting of positions 23, 262, 264 and 298. The undetermined amino acid in the sequence is indicated by “X” in SEQ ID NO: 1.

According to a second aspect of the invention, the TdT variant according to the invention comprises an amino acid sequence at least 70% identical to SEQ ID NO: 2, preferably at least 75%, 80%, 85%, 90%, 95%, 96%, 97%, 98%, 99% identical to SEQ ID NO:2 and optionally less than 100% identical to SEQ ID NO: 2, wherein said amino acid sequence comprises at least one amino acid substitution with a substitute amino acid at position selected from the first group consisting of positions 23, 262, 264 and 298, or at functionally equivalent position of each position of said first group, wherein the positions are numbered by reference to the amino acid sequence set forth in SEQ ID NO: 2. In particular, the TdT variant according to the invention (i) is capable of synthesizing a nucleic acid fragment without a template and (ii) is capable of incorporating a 3′-O-modified nucleotide onto a nucleic acid fragment.

In a particular embodiment, said amino acid sequence can comprise more than one amino acid substitution, preferably at least two, or three selected from the group consisting of positions 23, 262, 264 and 298. Said TdT variants are capable of synthesizing a DNA strand and/or an RNA strand.

In another embodiment, the TdT variant according to the invention has an amino acid sequence at least 70% identical to SEQ ID NO: 1, preferably at least 75%, 80%, 85%, 90%, 95%, 96%, 97%, 98%, 99% identical to SEQ ID NO:1 and optionally less than 100% identical to SEQ ID NO: 1, wherein said amino acid sequence comprises at least one amino acid substitution with a substitute amino acid at position selected from the group consisting of positions 23, 262, 264 and 298, or at functionally equivalent position of each position of said first group, wherein the positions are numbered by reference to the amino acid sequence set forth in SEQ ID NO: 1. Said TdT variant having an amino acid sequence at least 70% identical to SEQ ID NO: 1 comprises at least one undetermined amino acid at position selected from the first group consisting of positions 23, 262, 264 and 298, or at functionally equivalent position of each position of said first group. The undetermined amino acid in the sequence is indicated by “X” in SEQ ID NO: 1.

According to a second aspect of the invention, the TdT variant according to the invention comprises an amino acid sequence at least 70% identical to SEQ ID NO: 8, preferably at least 75%, 80%, 85%, 90%, 95%, 96%, 97%, 98%, 99% identical to SEQ ID NO:8 and optionally less than 100% identical to SEQ ID NO: 8, wherein said amino acid sequence comprises at least one amino acid replacement with a replacing amino acid at position selected from the second group consisting of positions 4, 243, 245 and 279, or at functionally equivalent position of each position of said second group, wherein the positions are numbered by reference to the amino acid sequence set forth in SEQ ID NO: 8. In particular, the TdT variant according to the invention (i) is capable of synthesizing a nucleic acid fragment without a template and (ii) is capable of incorporating a 3′-O-modified nucleotide onto a nucleic acid fragment.

In a particular embodiment, said amino acid sequence can comprise more than one amino acid replacement, preferably at least two, or three selected from the second group consisting of positions 4, 243, 245 and 279. Said TdT variants are capable of synthesizing a DNA strand and/or an RNA strand.

In another embodiment, the TdT variant according to the invention has an amino acid sequence at least 70% identical to SEQ ID NO: 7, preferably at least 75%, 80%, 85%, 90%, 95%, 96%, 97%, 98%, 99% identical to SEQ ID NO:7 and optionally less than 100% identical to SEQ ID NO: 7, wherein said amino acid sequence comprises at least one amino acid replacement with a replacing amino acid at position selected from the group consisting of positions 4, 243, 245 and 279, or at functionally equivalent position of each position of said first group, wherein the positions are numbered by reference to the amino acid sequence set forth in SEQ ID NO: 7. Said TdT variant having an amino acid sequence at least 70% identical to SEQ ID NO: 7 comprises at least one undetermined amino acid at position selected from the second group consisting of positions 4, 243, 245 and 279, or at functionally equivalent position of each position of said second group. The undetermined amino acid in the sequence is indicated by “X” in SEQ ID NO: 7.

In another embodiment, the TdT variant comprises the amino acid sequence as set forth in SEQ ID NO: 2, wherein said amino acid sequence comprises at least one amino acid substitution with an amino acid at a position corresponding to one residue selected from the group consisting of C23, V262, T264 and 1298, wherein the positions are numbered by reference to the amino acid sequence set forth in SEQ ID NO: 2. For example, residue C23 corresponds to Cysteine amino acid at position 23 before substitution.

In another embodiment, the TdT variant comprises an amino acid sequence at least 70% identical to SEQ ID NO: 2, wherein said amino acid sequence comprises at least one amino acid substitution with a substitute amino acid at a position corresponding to one residue selected from the group consisting of C23, V262, T264 and 1298, or at functionally equivalent position of each position of said group, wherein the positions are numbered by reference to the amino acid sequence set forth in SEQ ID NO: 2. For example, residue C23 corresponds to Cysteine amino acid at position 23 before substitution.

In another embodiment, the TdT variant comprises an amino acid sequence at least 70% identical to SEQ ID NO: 2, wherein said amino acid sequence comprises at least one amino acid replacement with a replacing amino acid at a position corresponding to one residue selected from the group consisting of C4, V243, T245 and 1279, or at functionally equivalent position of each position of said group, wherein the positions are numbered by reference to the amino acid sequence set forth in SEQ ID NO: 8. For example, residue C4 corresponds to Cysteine amino acid at position 23 before replacement. According to anyone of previous embodiments, substitution described above correspond to TdT residues located within the flexible loop L1-protein region that is involved in dNTP and iDNA binding, but also within the core of the catalytic domain-protein region that is involved in chemical step of reaction (nucleotidylexotransferase activity) and also located in C-term domain-protein region that is involved in iDNA binding and overall folding of TdT. Said substitutions specific position, in particular in the flexible loop L1, improves reaction yields due to better alignment between TdTs 3′OH of iDNA to the alpha phosphate of dNTP.

In some embodiments, the TdT variant according to the invention comprises the amino acid sequence as set forth in SEQ ID NO: 2 or a functionally equivalent sequence as set forth in SEQ ID NO: 2, wherein said amino acid sequence comprises at least one amino acid substitution with an amino acid at position selected from the group consisting of positions 23, 262, 264 and 298, wherein the positions are numbered by reference to the amino acid sequence set forth in SEQ ID NO: 2.

In the context of the invention, the TdT variant can be from any species or be a chimeric protein. By “chimeric protein”, is meant that portions of the variant are from at least 2 different species. Said chimeric protein is formed by the addition, and in particular fusion or conjugation, of one or more predetermined sequences of a protein of one species and at least one another predetermined sequence of a second species which is a member of the polX family, in particular a TdT. Preferably, the TdT variant is a chimeric protein, more preferably the chimeric protein comprises portions from 2 different species, in particular a predetermined sequence from mouse and a predetermined sequence from bovine.

In a particular embodiment, the TdT variant according to the invention comprises an amino acid sequence having at least 70%, 75%, 80%, 85%, 90% of identity or homology to the amino acid sequence as set forth in SEQ ID NO: 1 or in SEQ ID NO: 2, preferably at least 95%, 96%, 97%, 98%, 99% and less than 100% identity with the sequence according to SEQ ID NO: 1 or in SEQ ID NO: 2.

In another particular embodiment, the TdT variant according to the invention comprises an amino acid sequence having at least 70%, 75%, 80%, 85%, 90% of identity or homology to the amino acid sequence selected from the group consisting in SEQ ID NO: 3, SEQ ID NO: 4, and SEQ ID NO: 5, more preferably at least 95%, 96%, 97%, 98%, 99% and less than 100% identity with the sequence selected from the group consisting in SEQ ID NO: 3, SEQ ID NO: 4, and SEQ ID NO: 5.

In a particular embodiment, the at least one amino acid substitution with the substitute amino acid at position selected from the group consisting of positions 23, 262, 264 and 298 is selected from the group consisting of R, V, A, L, K, Q, T and G. More preferably, the substituted amino acid is at position 298, the positions indicated being determined by alignment with SEQ ID NO: 1 or SEQ ID NO: 2.

In a particular embodiment, the at least one amino acid replacement with the replacing amino acid at position selected from the group consisting of positions 4, 243, 245 and 279 is selected from the group consisting of R, V, A, L, K, Q, T and G. More preferably, the replaced amino acid is at position 279, the positions indicated being determined by alignment with SEQ ID NO: 7 or SEQ ID NO: 8.

In some embodiments, the amino acid sequence as set forth in SEQ ID NO: 1 or SEQ ID NO: 2 comprises at least two substitutions at positions selected from the group consisting of positions 23, 262, 264 and 298. Preferably, the first substitution is at position 298 and the second is at position selected from the group consisting of positions 23, 262, and 264.

In some embodiments, the amino acid sequence at least 70% identical to SEQ ID NO: 1 or SEQ ID NO: 2 comprises at least two substitutions at positions selected from the group consisting of positions 23, 262, 264 and 298. Preferably, the first substitution is at position 298 and the second is at position selected from the group consisting of positions 23, 262, and 264. I

In some embodiments, the amino acid sequence at least 70% identical to SEQ ID NO: 7 or SEQ ID NO: 8 comprises at least two replacements at positions selected from the group consisting of positions 4, 243, 245 and 279. Preferably, the first substitution is at position 279, and the second is at position selected from the group consisting of positions 4, 243, and 245.

In a particular embodiment the at least two substitutions are selected from the group consisting of R, V, A, L, K, Q, T and G.

In a particular embodiment the at least two replacements are selected from the group consisting of R, V, A, L, K, Q, T and G. In some embodiments, the amino acid sequence as set forth in SEQ ID NO: 1 or SEQ ID NO: 2 comprises at least three substitutions at positions selected from the group consisting of positions 23, 262, 264 and 298. Preferably, the first substitution is at position 298, the second is at position 23 or 264 and the third is also at position 23 or 264.

In some embodiments, the amino acid sequence at least 70% identical to SEQ ID NO: 1 or SEQ ID NO: 2 comprises at least three substitutions at positions selected from the group consisting of positions 23, 262, 264 and 298. Preferably, the first substitution is at position 298, the second is at position 23 or 264 and the third is also at position 23 or 264.

In some embodiments, the amino acid sequence at least 70% identical to SEQ ID NO: 7 or SEQ ID NO: 8 comprises at least three replacements at positions selected from the group consisting of positions 4, 243, 245 and 279. Preferably, the first replacement is at position 279, the second replacement is at position 4 or 245 and the third replacement is also at position 4 or 245.

In a particular embodiment, the three substitutions are selected from the group consisting of R, V, A, L, K, Q, T and G.

In a particular embodiment, the three replacements are selected form the group consisting of R, V, A, L, K, Q, T and G.

In a particular embodiment, the amino acid sequence of the TdT variant according to the invention as set forth in SEQ ID NO: 2 comprises at least one amino acid substitution selected from 1298V/T/L. By “1298V/T/L”, this means that Isoleucine is substituted at position 298 by the amino acid selected from Valine (V), Threonine (T) and Leucine (L). Preferably, the at least one amino acid substitution is 1298/V.

In some embodiments, the amino acid sequence at least 70% identical to SEQ ID NO: 2 comprises at least one amino acid substitution selected from 1298V/T/L.

In some embodiments, the amino acid sequence at least 70% identical to SEQ ID NO: 8 comprises at least one amino acid replacement selected from 1279V/T/L.

In another particular embodiment, the amino acid sequence of the TdT variant as set forth in SEQ ID NO: 2 comprises at least one amino acid substitution selected from T264R/K/Q. By “T264R/K/Q”, this means that Threonine is substituted at position 264 by the amino acid selected from Arginine (R), Leucine (K) and Glutamine (Q). Preferably, the at least one amino acid substitution is T264/R.

In some embodiments, the amino acid sequence at least 70% identical to SEQ ID NO: 2 comprises at least one amino acid substitution selected from T264R/K/Q.

In some embodiments, the amino acid sequence at least 70% identical to SEQ ID NO: 8 comprises at least one amino acid replacement selected from T245R/K/Q.

In another particular embodiment, the amino acid sequence of the TdT variant as set forth in SEQ ID NO: 2 comprises at least one amino acid substitution selected from V262A/T/G. By “V262A/T/G”, this means that Valine is substituted at position 262 by the amino acid selected from Alanine (A), Threonine (T) and Glycine (G). Preferably, the at least one amino acid substitution is V262A.

In some embodiments, the amino acid sequence at least 70% identical to SEQ ID NO: 2 comprises at least one amino acid substitution selected from V262A/T/G.

In some embodiments, the amino acid sequence at least 70% identical to SEQ ID NO: 8 comprises at least one amino acid replacement selected from V243A/T/G.

In another particular embodiment, the amino acid sequence of the TdT variant as set forth in SEQ ID NO: 2 comprises at least one amino acid substitution selected from C23A/T. By “C23A/T”, this means that Cysteine is substituted at position 23 by the amino acid Alanine (A) or Threonine (T).

Preferably, the at least one amino acid substitution is C23A.

In some embodiments, the amino acid sequence at least 70% identical to SEQ ID NO: 2 comprises at least one amino acid substitution selected from C23A/T.

In some embodiments, the amino acid sequence at least 70% identical to SEQ ID NO: 8 comprises at least one amino acid replacement selected from C4A/T.

In some embodiments, the amino acid sequence of the TdT variant as set forth in SEQ ID NO: 2 comprises at least two amino acid substitution selected from C23A/T, V262A/T/G, T264R/K/Q and 1298V/T/L, preferably the two amino acid substitution are T264R/K/Q and 1298V/T/L, or C23A/T and 1298V/T/L, or V262A/T/G and 1298V/T/L.

In some embodiments, the amino acid sequence at least 70% identical to SEQ ID NO: 2 comprises at least two amino acid substitution selected from C23A/T, V262A/T/G, T264R/K/Q and 1298V/T/L, preferably the two amino acid substitution are T264R/K/Q and 1298V/T/L, or C23A/T and 1298V/T/L, or V262A/T/G and 1298V/T/L.

In some embodiments, the amino acid sequence at least 70% identical to SEQ ID NO: 8 comprises at least two amino acid substitution selected from C4A/T, V243A/T/G, T245R/K/Q and 1279V/T/L, preferably the two amino acid substitution are T245R/K/Q and 1279V/T/L, or C4A/T and 1279V/T/L, or V243A/T/G and 1279V/T/L.

In other preferred embodiment, the amino acid sequence of the TdT variant as set forth in SEQ ID NO: 2 comprises three amino acid substitution which are C23A/T, and T264R/K/Q and 1298V/T/L.

In some embodiments, the amino acid sequence at least 70% identical to SEQ ID NO: 2 comprises three amino acid substitution which are C23A/T, and T264R/K/Q and 1298V/T/L.

In some embodiments, the amino acid sequence at least 70% identical to SEQ ID NO: 8 comprises three amino acid substitution which are C4A/T, and T245R/K/Q and 1279V/T/L.

In some embodiments, said at least one amino acid substitution is at position 298.

In some embodiments, said at least one amino acid replacement is at position 279.

In some embodiments, the amino acid sequence at least 70% identical to SEQ ID NO: 2 comprises at least two substitutions at positions selected from the group consisting of positions 23, 262, 264 and 298.

In some embodiments, the amino acid sequence at least 70% identical to SEQ ID NO: 8 comprises at least two replacements at positions selected from the group consisting of positions 4, 243, 245 and 279.

In some embodiments, the amino acid sequence at least 70% identical to SEQ ID NO: 2 comprises at least three substitutions at positions selected from the group consisting of positions 23, 262, 264 and 298.

In some embodiments, the amino acid sequence at least 70% identical to SEQ ID NO: 8 comprises at least three replacements at positions selected from the group consisting of positions 4, 243, 245 and 279.

In a preferred embodiment the TdT variants of the invention are chimeric variants and are listed in Table 1 below.

TABLE 1

| SEQ | | | | | |
|---|---|---|---|---|---|
| ID | | | | | |
| NO | Animal | Substitutions | | | |
| 1 | Chimeric | 23 | 262 | 264 | 298 |
| 2 | Chimeric | C23 | V262 | T264 | 1298 |
| 3 | Chimeric | — | — | T264R/K/Q | I298V/T/L |
| 4 | Chimeric | C23A/T | — | T264R/K/Q | I298V/T/L |
| 5 | Chimeric | — | V262A/T/G | — | I298V/T/L |
| 7 | Chimeric | 4 | 243 | 245 | 279 |
| 8 | Chimeric | C4 | V243 | T245 | I279 |
| 9 | Chimeric | — | — | T245R/K/Q | I279V/T/L |
| 10 | Chimeric | C4A/T | — | T245R/K/Q | I279V/T/L |
| 11 | Chimeric | — | V243A/T/G | --- | I279V/T/L |

In some embodiments, the TdT variant comprises the amino acid sequence selected from the group consisting of SEQ ID NO:3, SEQ ID NO:4, SEQ ID NO:5, preferably the TdT variant having the amino acid sequence as set forth in SEQ ID NO:3, or SEQ ID NO:5, more preferably the TdT variant having the amino acid sequence as set forth in SEQ ID NO:5.

In some embodiments, the TdT variant comprises the amino acid sequence selected from the group consisting of SEQ ID NO:9, SEQ ID NO: 10, SEQ ID NO:11, preferably the TdT variant having the amino acid sequence as set forth in SEQ ID NO:9 or SEQ ID NO: 11, more preferably the TdT variant having the amino acid sequence as set forth in SEQ ID NO:11.

In some embodiments, the TdT variant comprising the amino acid sequence at least 70% identical to SEQ ID NO: 2 is at least 70% identical to, preferably at least 75%, 80%, 85%, 90%, 95%, 96%, 97%, 98%, 99% identical to and optionally less than 100% identical to, an amino acid sequence selected from the group consisting of SEQ ID NO:3, SEQ ID NO:4 and SEQ ID NO:5.

The TdT variant comprising the amino acid sequence at least 70% identical to SEQ ID NO: 8 is at least 70% identical to, preferably at least 75%, 80%, 85%, 90%, 95%, 96%, 97%, 98%, 99% identical to and optionally less than 100% identical to, an amino acid sequence selected from the group consisting of SEQ ID NO:9, SEQ ID NO: 10 and SEQ ID NO:11.

Said specific TdT variants according to the invention improve the quality of the DNA synthesis and then solve the technical problem. In particular, the TdT variants having the amino acid sequence as set forth in SEQ ID NO:3, SEQ ID NO:4 and SEQ ID NO:5 have a reduced deletion rate for Alanine from 0.55% to 0.36%, 0.32% and 0.40% per step, respectively compared to the deletion rate of the TdT variant having the amino acid sequence as set forth in SEQ ID NO:2. Same TdT variants reduces global synthesis error rate by 0.04%, 0.06% and 0.06% per step, respectively. Therefore, said specific TdT variants according to the invention are more stable compared to TdT variant having the amino acid sequence as set forth in SEQ ID NO:2.

Also in particular, the TdT variant having an amino acid sequence at least 70% identical to SEQ ID NO: 3, SEQ ID NO:4, SEQ ID NO:5, SEQ ID: 9, SEQ ID NO: 10 or SEQ ID NO: 11 has a reduced deletion rate for Alanine from 0.55% to 0.36%, 0.32% and 0.40% per step, respectively compared to the deletion rate of the TdT variant having the amino acid sequence as set forth in SEQ ID NO:2 or having the amino acid sequence set forth in SEQ ID NO: 8. Same TdT variants reduces global synthesis error rate by 0.04%, 0.06% and 0.06% per step, respectively. Therefore, said specific TdT variants according to the invention are more stable compared to TdT variant having the amino acid sequence as set forth in SEQ ID NO:2 or as set forth in SEQ ID NO: 8.

In another embodiment, the present invention relates to a nucleic acid coding for a variant of a DNA polymerase of the polX family, in particular a TdT capable of synthesizing a nucleic acid molecule without a template strand according to the present invention. The present invention also relates to an expression cassette of a nucleic acid according to the present invention. The invention further relates to a vector comprising a nucleic acid or an expression cassette according to the present invention. The vector can be selected from a plasmid or a viral vector.

The nucleic acid coding for the DNA polymerase variant can be DNA (cDNA or gDNA), RNA, a mixture of the two. It can be in single-strand form or in duplex form or a mixture of the two forms. It can comprise modified nucleotides comprising, for example, a modified bond, a modified purine or pyrimidine base, or a modified sugar. It can be prepared by any of the methods known to the person skilled in the art, including chemical synthesis, recombination, mutagenesis, etc.

The expression cassette comprises all the elements necessary for the expression of the TdT variant according to the present invention, in particular the elements necessary for transcription and translation in the host cell. The host cell can be prokaryotic or eukaryotic. In particular, the expression cassette comprises a promoter and a terminator, optionally an amplifier. The promoter can be prokaryotic or eukaryotic. The following are examples of preferred prokaryotic promoters: LacI, LacZ, placT, ptac, PARA, pBAD, the bacteriophage T3 or T7 RNA polymerase promoters, the polyhydrin promoter, the lambda phage PR or PL promoter. The following are examples of preferred eukaryotic promoters: the early CMV promoter, the HSV thymidine kinase promoter, the early or late SV40 promoter, the murine metallothionein-L promoter, and LTR regions of certain retroviruses. In general, for the selection of an appropriate promoter, the person skilled in the art can advantageously refer to the work by Sambrook et al. (1989) or to the techniques described by Fuller et al. (1996; Immunology in Current Protocols in Molecular Biology).

The present invention relates to a vector carrying a nucleic acid or an expression cassette coding for a TdT variant according to the present invention. The vector is preferably an expression vector, i.e., it comprises the elements necessary for the expression of the variant in the host cell. The host cell can be a prokaryote, for example, E. coli, or a eukaryote. The eukaryote can be a lower eukaryote such as a yeast (for example, P. pastoris or K. lactis) or a fungus (for example, of the Aspergillus genus) or a higher eukaryote such as an insect cell (Sf9 or Sf21, for example), a mammalian cell or a plant cell. The cell can be a mammalian cell, for example, COS (green monkey cell line) (for example, COS 1 (ATCC CRL-1650), COS 7 (ATCC CRL-1651), CHO (U.S. Pat. Nos. 4,889,803; 5,047,335, CHO-K1 (ATCC CCL-61)), murine cells and human cells. In a particular embodiment, the cell is non-human and non-embryonic. The vector can be a plasmid, a phage, a phagemid, a cosmid, a virus, a YAC, a BAC, an Agrobacterium pTi plasmid, etc. The vector can preferably comprise one or more elements selected from a replication origin, a multiple cloning site and a selection gene. In a preferred embodiment, the vector is a plasmid. The following are non-exhaustive examples of prokaryotic vectors: pQE70, pQE60, pQE-9 (Qiagen), pbs, pD10, phagescript, psiX174, pbluescrip SK, pbsks, pNH8A, pNH16A, pNH18A, pNH46A (Stratagene); ptrc99a, pKK223-3, pKK233-3, pDR540, pBR322, and pRIT5 (Pharmacia), pET (Novagen). The following are non-exhaustive examples of eukaryotic vectors: pWLNEO, pSV2CAT, pPICZ, pcDNA3.1 (+) Hyg (Invitrogen), pOG44, pXT1, pSG (Strategene); pSVK3, pBPV, pCI-neo (Stratagene), pMSG, pSVL (Pharmacia); and pQE-30 (QLAexpress). The viral vectors can be in a non-exhaustive manner adenoviruses, AAV, HSV, lentiviruses, etc. Preferably, the expression vector is a plasmid or a viral vector.

The sequence coding for the TdT variant according to the present invention may or may not comprise a signal peptide. In the case in which it does not comprise a signal peptide, a methionine can optionally be added to the N-terminal end. In another alternative, a heterologous signal peptide can be introduced. This heterologous signal peptide can be derived from a prokaryote such as E. coli or from a eukaryote, in particular a mammalian cell, an insect cell, or a yeast.

The present invention relates to the use of a polynucleotide, of an expression cassette or of a vector according to the present invention for transforming or transfecting a cell. The present invention relates to a host cell comprising a nucleic acid, an expression cassette or a vector coding for a TdT variant and to its use for producing a TdT variant according to the present invention. The term “host cell” encompasses the daughter cells resulting from the culture or from the growth of this cell. In a particular embodiment, the cell is non-human and non-embryonic.

The present invention also relates to a method for producing a TdT variant of the invention, comprising the transformation or transfection of a cell by a polynucleotide, an expression cassette or a vector according to the present invention; the culturing of the transfected/transformed cell; and the harvesting of the TdT variant produced by the cell. In an alternative embodiment, a method for producing a TdT variant according to the present invention comprises the provision of a cell comprising a polynucleotide, an expression cassette or a vector according to the invention; the culturing of the transfected/transformed cell; and the harvesting of the TdT variant produced by the cell. In particular, the cell can be transformed/transfected in a transient or stable manner by the nucleic acid coding for the variant. This nucleic acid can be contained in the cell in the form of an episome or in chromosomal form. The methods for producing recombinant proteins are well known to the person skilled in the art.

TdT variants may be operably linked to a linker moiety including a covalent or non-covalent bond; amino acid tag (e.g., poly-amino acid tag, poly-His tag, 6His-tag, or the like); chemical compound (e.g., polyethylene glycol); protein-protein binding pair (e.g., biotin-avidin); affinity coupling; capture probes; or any combination of these. The linker moiety can be separate from, or part of a TdT variant. An exemplary His-tag for use with modified TdT variants of the invention is MASSHHHHHHSSGSEKKIS-(SEQ ID NO: 6). The tag-linker moiety does not interfere with the nucleotide binding activity, or catalytic activity of the TdT variants and allow easy purification and isolation of the TdT variants.

The TdT variants according to the present invention are particularly advantageous for the synthesis of nucleic acids without a template strand. More particularly, the variants according to the invention have more flexible Loop 1 that is more suitable for accommodation of modified nucleotides exhibiting greater steric hindrance than the natural nucleotides. Also, T264R mutation in SEQ ID NO: 2 or T245R in SEQ ID NO: 8 improves the interaction between TdT and the DNA initiator.

Thus, the invention also relates to a use of a TdT variant according to the present invention for synthesizing a nucleic acid molecule without a template strand, from 3′-OH modified nucleotides, and in particular those described in the application WO2016034807.

In another aspect, the invention relates to a kit, in particular for the enzymatic synthesis of a nucleic acid molecule without a template strand, said kit comprises:
a. at least one TdT variant according to anyone of the above described embodiments,
b. at least one 3′-O-modified nucleoside triphosphate, and
c. optionally at least one initiator.

In particular, the modified nucleoside triphosphate is a protecting group. This protecting group allows to block/protect the 3′-OH and therefore to prevent reaction with other nucleoside. Therefore, in particular, the kit comprises at least one 3′-O-protected nucleoside triphosphate.

In a particular embodiment, the kit of the invention comprises at least two TdT variants, preferably at least three TdT variant. When, the kit comprises more than one TdT variant, then the TdT variants in the kit could be different from each other. For example, it can be a mix of TdT variants of the invention.

In some embodiments, the kit comprises a first TdT variant comprising an amino acid sequence at least 70% identical to SEQ ID NO: 3, preferably at least 75%, 80%, 85%, 90%, 95%, 96%, 97%, 98%, 99% identical to SEQ ID NO: 3 and optionally less than 100% identical to SEQ ID NO: 3, and a second TdT variant comprising an amino acid sequence at least 70% identical to SEQ ID NO: 5, preferably at least 75%, 80%, 85%, 90%, 95%, 96%, 97%, 98%, 99% identical to SEQ ID NO: 5 and optionally less than 100% identical to SEQ ID NO: 5.

In a more preferred embodiment, the kit of the invention comprises a first TdT variant comprises the amino acid sequence as set forth in SEQ ID NO:3 and a second variant comprises the amino acid sequence as set forth in SEQ ID NO:5.

In some embodiments, the kit of the invention comprises a first TdT variant comprising an amino acid sequence at least 70% identical to SEQ ID NO: 9, preferably at least 75%, 80%, 85%, 90%, 95%, 96%, 97%, 98%, 99% identical to SEQ ID NO: 9 and optionally less than 100% identical to SEQ ID NO: 9, and a second TdT variant comprising an amino acid sequence at least 70% identical to SEQ ID NO: 11, preferably at least 75%, 80%, 85%, 90%, 95%, 96%, 97%, 98%, 99% identical to SEQ ID NO: 11, and optionally less than 100% identical to SEQ ID NO: 11.

In a more preferred embodiment, the kit of the invention comprises a first TdT variant comprises the amino acid sequence as set forth in SEQ ID NO: 9 and a second TdT variant comprises the amino acid sequence as set forth in SEQ ID NO: 11.

Said TdT variants and said kits of the invention is particularly suitable for the enzymatic synthesis of a polynucleotide, i.e. nucleic acid molecule, without a template strand.

Therefore, the invention also relates to a method for the enzymatic synthesis of a nucleic acid molecule without a template strand, according to which a primer strand is brought in contact with at least one nucleotide, preferably a 3′-OH modified nucleotide, in the presence of a TdT variant according to the invention.

Thus, the invention relates to a method of synthesizing a polynucleotide, optionally having a predetermined sequence, wherein the method comprising the steps of:
a. providing at least one initiator having a 3′-terminal nucleotide which has a free 3′-hydroxyl,
b. contacting under elongation conditions said at least one initiator having a free 3′-O-hydroxyl with a 3′-O-blocked nucleoside triphosphate and a TdT variant according to anyone of the above described embodiments, so that said at least one initiator is elongated by incorporation of a 3′-O-blocked nucleoside triphosphate to form a 3′-O-blocked elongated fragment, and
c. deblocking the elongated fragment to form an elongated fragment having a free 3′-hydroxyl, and
d. repeating steps b. and c. by contacting under elongation conditions the elongated fragment obtained in step c., until the polynucleotide is formed.

In a preferred embodiment, the TdT variants according to the invention can be used to carry out the synthesis method described in the application WO2015/159023 incorporated by reference herein.

Other features and advantages of the invention will be more clearly from the following examples and results which are of course non-limiting.

### Examples

### Example 1—TdT Variants Construction, Expression and Screening

### TdT Variant Construction

TdT variants of the invention were created using MEGAWHOP cloning method as disclosed by Miyazaki K. MEGAWHOP cloning: a method of creating random mutagenesis libraries via megaprimer PCR of whole plasmids. Methods Enzymol. 2011; 498:399-406. Doi: 10.1016/B978-O-12-385120-8.00017-6. PMID: 21601687.

Megaprimers were made by PCR with a pair of primers where at least one primer was mutagenic (containing degenerate codon or predesigned mutation) according to the usual PCR amplification and molecular biology techniques. Purified megaprimers were combined with TDT circular backbone plasmid (pet28 vector) in a second PCR step. After digestion with DpnI, PCR products were electroporated in E. Cloni 10G cells (Lucigen). Transformants were selected on 2YT-agar plates supplemented with 50 mg/L Kanamycin. Plasmids encoding the TdT variants were prepared either directly from transformation plate bacteria carpets or from overnight liquid cultures (2YT supplemented with 0.5% glucose and 50 mg/L Kanamycin).

### Expression and Screening

For screening, plasmids encoding for TdT variants were retransformed in the commercial E. coli strains BL21 (DE3) (Novagen). The colonies that were capable of growing in kanamycin petri dishes were isolated and labeled. Individual variants of TdT were produced, expressed and purified in 96 well format using Ni-NTA chromatography as described by Ybert et al, American patent application US 2020/0002690. Protein amounts were determined by absorbance at 280 nm.

### Example 2—Study of the Activity of the TdT Variants, in Particular Duplex and Hairpin Activity

The activity of different TdT variants according to the invention was determined by the following test. The results were compared to those obtained with the TdT of SEQ ID NO: 2 (reference) from which each of the variant is derived.

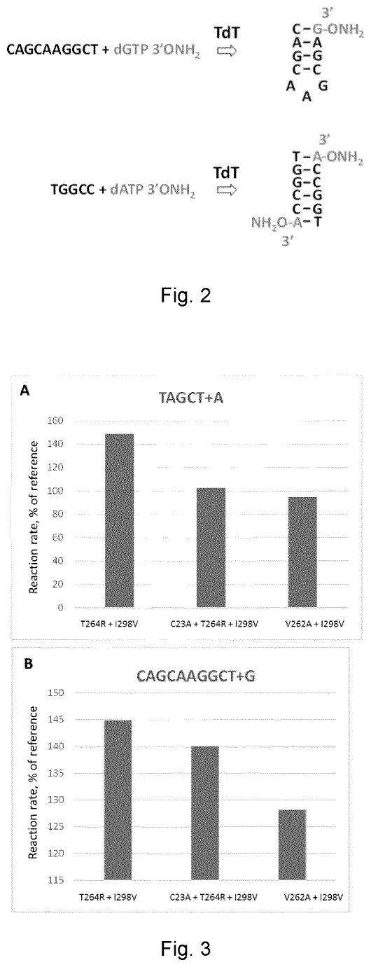

This study monitors TdT kinetics in the incorporation of 3′ terminated dNTP as described in international patent application number WO2020/099451. Inventors have developed a short initiator DNA (iDNA) substrates that transition from single to double stranded DNA (dsDNA) upon the incorporation of a single base (TGGCC for +A reaction and CAGCAAGGCT for +G reaction). Depending on application, two types of iDNA were applied: Hairpin-DNA that mainly forms intramolecular dsDNA or Duplex-DNA that forms symmetric dsDNA comprised of two molecules (see FIG. 2). In duplex assay, energetic effects of a single incorporated base are doubled, and this is crucial to monitor+A or +T reactions that create easily melting A=T pairs.

Therefore, in such system, formation of dsDNA reflects the enzymatic activity of TdT and it can be monitored in real time by measuring fluorescence of dsDNA specific intercalating dye (Ethidium bromide, GelRed, SYBR Green). Specific activity of TdT variants was determined as an initial rate of GelRed fluorescence increase in presence of 500 μM 3′ONH2 dNTP, 10 UM of iDNA and 30 nM of TdT.

### Results

OP2 represents the purity of the enzymatically made DNA determined by capillary electrophoresis. E12 stands for TGTTCCGGAAGAGCAACCTG DNA sequence synthetized atop of AACTACCTGTACCGGC DNA attached to solid support. Q21 stands for CGCACGCTAC DNA sequence synthetized atop of GTATGGCGCGATGACTCG DNA attached to solid support. Tm stands for melting temperature of pure TdT variants, determined from thermal shift assay using SYPRO Orange dye. Deletion rates are averages from our standard 24 primer set which are about 50 bases long on average.

Results are detailed in Table 2 below.

TABLE 2

| SEQ ID NO | | | | 2 |
|---|---|---|---|---|
| (TdT | | | | (refer- |
| variant) | 3 | 4 | 5 | ence) |
| Mutation | T264R + | C23A + | V262A + | |
| | I298V | T264R + | I298V | |
| | | I298V | | |
| TAGCT + A rate | 149 | 102 | 95 | 100 |
| (% of ′ref) | | | | |
| TGGCC + A rate | 218 | 176 | 134 | 100 |
| (% of ′ref) | | | | |
| CAGCAAGGCT + | 145 | 140 | 128 | 100 |
| G rate (% of | | | | |
| ′ref) | | | | |
| OP2 purity, e12 | 65 | 64 | 65 | 77 |
| seq (%) | | | | |
| OP2 purity, q21 | 80 | 82 | 76 | 77 |
| seq (%) | | | | |
| TM, ° C. (TSA) | 60 | 53 | 62 | 61 |
| ngs_+A deletion | 0.36 | 0.32 | 0.4 | 0.55 |
| rate (%) | | | | |
| ngs_global | 0.28 | 0.24 | 0.24 | 0.32 |
| deletion rate (%) | | | | |

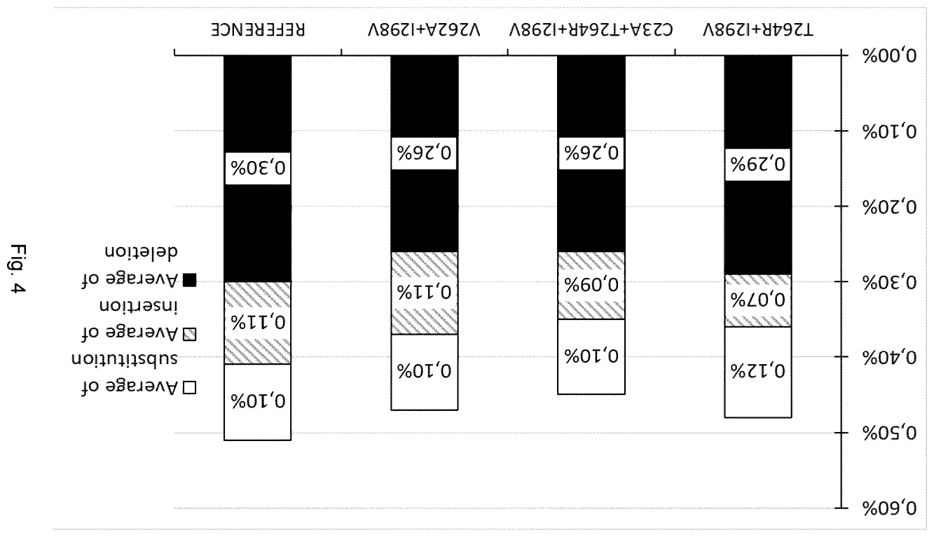

Activity and synthesis performance of TdT variant having SEQ ID NO:3 or SEQ ID NO:4 or SEQ ID NO: 5, respectively variant having mutation T264R+1298V, or C23A+T264R+1298V, or V262A+1298V are represented on FIG. 3. Plot in panel A shows enzymatic rates for +A reaction in TAGCT+A duplex test and plot in panel B indicates enzymatic rates for +G reaction with CAGCAAGGCT+G hairpin.

Therefore, the TdT variant having the amino acid sequence as set forth in SEQ ID NO:3, SEQ ID NO: 4 and SEQ ID NO:5 reduced deletion rate for Alanine from 0.55% to 0.36%, 0.32% and 0.40% per step, respectively compared to the deletion rate of the TdT variant having the amino acid sequence as set forth in SEQ ID NO:2. Same TdT variants reduces global synthesis error rate by 0.04%, 0.06% and 0.06% per step, respectively. Therefore, said specific TdT variants according to the invention produce better quality DNA strand.

### Example 3—Comparative Study

This study investigates the average of substitution, insertion, deletion for sequences synthetized by TdT variant of SEQ ID NO: 3, SEQ ID NO: 4, or SEQ ID NO: 5 compared to those obtained with the TdT of SEQ ID NO: 2.

24 sequences are synthesized in duplicates for each TdT variant. Syntheses are performed at 37° C. with 20 μM Enzyme, 2 mM CoCl₂, 500 μM ONH2-nucleotides, 750 pmols resin (10 μM iDNA) at 20% DMSO, 50 mM NaCl in 0.5 M Cacodylate buffer pH 7.4. Each synthesis includes TdT of SEQ ID NO: 2 as control. The percentage refers to the percentage of perfect reads compared to the reference sequence).

The 24 sequences synthesized by each TdT variant were sequenced using an iSeq 100 System sequencer from Illumina, Inc. The percentage of deletion, insertion and substitution are calculated by comparing the sequences obtained from the sequencer and the targeted sequences.

The results are detailed in FIG. 4.

Regarding the average of deletion, the TdT variant of SEQ ID NO: 3, SEQ ID NO: 4, and SEQ ID NO: 5 are all better, in particular TdT variant of SEQ ID NO: 4 and SEQ ID NO: 5 compared to TdT of SEQ ID NO: 2. The average of insertion is also reduced than TdT of SEQ ID NO:2. Therefore, TdT variants according to the invention produce DNA strand with less misincorporations, the technical problem is thus well solved by the TdT of the invention.

1. A terminal deoxynucleotidyl transferase (TdT) variant comprising an amino acid sequence at least 70% identical to SEQ ID NO: 2, wherein said amino acid sequence comprises at least one amino acid substitution with a substitute amino acid at position selected from a first group consisting of positions 23, 262, 264 and 298, or at functionally equivalent position of each position of said first group, wherein the positions are numbered by reference to the amino acid sequence set forth in SEQ ID NO: 2 or an amino acid sequence at least 70% identical to SEQ ID NO: 8, wherein said amino acid sequence comprises at least one amino acid replacement with a replacing amino acid at position selected from a second group consisting of positions 4, 243, 245 and 279, or at functionally equivalent position of each position of said second group, wherein the positions are numbered by reference to the amino acid sequence set forth in SEQ ID NO: 8.

2. The TdT variant according to claim 1, wherein the substitute amino acid or the replacing amino acid is selected from the group consisting of R, V, A, L, K, Q, T and G.

3. The TdT variant according to claim 1, wherein said at least one amino acid substitution is at position 298 or said at least one amino acid replacement is at position 279.

4. The TdT variant according to claim 1, wherein the amino acid sequence at least 70% identical to SEQ ID NO: 2 comprises at least two substitutions at positions selected from the group consisting of positions 23, 262, 264 and 298 or the amino acid sequence at least 70% identical to SEQ ID NO: 8 comprises at least two replacements at positions selected from the group consisting of positions 4, 243, 245 and 279.

5. The TdT variant according to claim 1, wherein the amino acid sequence at least 70% identical to SEQ ID NO: 2 comprises at least three substitutions at positions selected from the group consisting of positions 23, 262, 264 and 298 or the amino acid sequence at least 70% identical to SEQ ID NO: 8 comprises at least three replacements at positions selected from the group consisting of positions 4, 243, 245 and 279.

6. The TdT variant according to claim 1, wherein the amino acid sequence at least 70% identical to SEQ ID NO: 2 comprises at least one amino acid substitution selected from 1298V/T/L or the amino acid sequence at least 70% identical to SEQ ID NO: 8 comprises at least one amino acid replacement selected from I279V/T/L.

7. The TdT variant according to claim 1, wherein the amino acid sequence at least 70% identical to SEQ ID NO: 2 comprises at least one amino acid substitution selected from T264R/K/Q or the amino acid sequence at least 70% identical to SEQ ID NO: 8 comprises at least one amino acid replacement selected from T245R/K/Q.

8. The TdT variant according to claim 1, wherein the amino acid sequence at least 70% identical to SEQ ID NO: 2 comprises at least one amino acid substitution selected from V262A/T/G or the amino acid sequence at least 70% identical to SEQ ID NO: 8 comprises at least one amino acid replacement selected from V243A/T/G.

9. The TdT variant according to claim 1, wherein the amino acid sequence at least 70% identical to SEQ ID NO: 2 comprises at least one amino acid substitution selected from C23A/T or the amino acid sequence at least 70% identical to SEQ ID NO: 8 comprises at least one amino acid replacement selected from C4A/T.

10. The TdT variant according to claim 1, wherein the amino acid sequence at least 70% identical to SEQ ID NO: 2 is at least 70% identical to an amino acid sequence selected from the group consisting of SEQ ID NO:3, SEQ ID NO: 4 and SEQ ID NO:5 or the amino acid sequence at least 70% identical to SEQ ID NO: 8 is at least 70% identical to an amino acid sequence selected from the group consisting of SEQ ID NO: 9, SEQ ID NO: 10 and SEQ ID NO:11.

11. A kit comprising:
a. at least one TdT variant according to claim 1,
b. at least one 3′-O-modified nucleoside triphosphate, and
c. optionally at least one initiator.

12. The kit according to claim 11, wherein it comprises two TdT variants.

13. The kit according to claim 12, wherein the first TdT variant an amino acid sequence at least 70% identical to SEQ ID NO: 3 and the second variant comprises an amino acid sequence at least 70% identical to SEQ ID NO: 5 or the first TdT variant an amino acid sequence at least 70% identical to SEQ ID NO: 9 and the second variant comprises an amino acid sequence at least 70% identical to SEQ ID NO: 11.

14. A method of synthesizing a polynucleotide, the method comprising the steps of:
a. providing at least one initiator having a 3′-terminal nucleotide which has a free 3′-hydroxyl,
b. contacting under elongation conditions the at least one initiator having free 3′-O-hydroxyl with a 3′-O-blocked nucleoside triphosphate and a TdT variant according to claim 1, so that said at least one initiator is elongated by incorporation of a 3′-O-blocked nucleoside triphosphate to form a 3′-O-blocked elongated fragment, and
c. deblocking the elongated fragment to form an elongated fragment having a free 3′-hydroxyl, and
d. repeating steps b. and c. by contacting under elongation conditions said at least elongated fragment obtained in step c., until the polynucleotide is formed.
